(19) United States
(12) Patent Application Publication
(10) Pub. No.: US 2014/0182399 A1
(43) Pub. Date: Jul. 3, 2014

(54) MULTI-FUNCTION BIOLOGICAL SENSING DEVICE

(71) Applicant: APEX BIOTECHNOLOGY CORP., Hsinchu (TW)

(72) Inventors: Tien-Chuan Ku, Hsinchu County (TW); Ching-Yuan Chu, Hsinchu (TW)

(73) Assignee: APEX BIOTECHNOLOGY CORP., Hsinchu (TW)

(21) Appl. No.: 13/888,384

(22) Filed: May 7, 2013

(30) Foreign Application Priority Data
Dec. 27, 2012 (TW) 101225332

(51) Int. Cl. G01N 33/48

(52) U.S. Cl. CPC G01N 33/48
USPC 73/866

(57) ABSTRACT

A multi-function biological sensing device including a multi-function card, a multi-function card slot, a first multi-function module, and a second multi-function module is provided. The multi-function card includes a first pin set. The multi-function card slot is configured to receive the multi-function card and includes a second pin set. The first multi-function module is disposed on the multi-function card and electrically connected to the first pin set. The second multi-function module is disposed on the multi-function card slot and electrically connected to the second pin set. Accordingly, the first multi-function module and the second multi-function module perform an electrical connection via the first pin set and the second pin set so as to provide at least one additional function.

### CROSS-REFERENCE TO RELATED APPLICATION

This application claims the priority benefit of Taiwan application serial no. 101225332, filed on Dec. 27, 2012. The entirety of the above-mentioned patent application is hereby incorporated by reference herein and made a part of this specification.

### BACKGROUND OF THE INVENTION

1. Field of the Invention

The present invention generally relates to a biological sensing device, in particular, to a multi-function biological sensing device with additional functions provided by multi-function modules on a multi-function card and a multi-function card slot.

2. Description of Related Art

The current biological sensing devices available on market normally include a sample strip slot and a password card slot. The sample strip slot is configured to allow sample strips with biological characteristics to be inserted therein so as to obtain related information such as blood glucose, uric acid, cholesterol, pH value through analyzing the biological characteristics on the sample strips. The password card slot is configured to allow the password card to be inserted therein so as to provide information such as parameters or coefficients related to the sample strip. For example, a biological sensor with a memory card slot is disclosed in U.S. Pat. No. 5,366,609. Such memory card slot may receive a removable read-only memory (ROM) password module so as to select a suitable parameter to adjust a sample strip from the password module. However, in such approach, two slots with different functions are required to be disposed on the biological sensing device and take more space. Therefore, as an improvement, Taiwan Patent No. M262189 proposes a concept of integrating a sample strip and a password card so as to reduce space from disposing a plurality of slots on the same biological sensing device.

With the development of medical technology, sensing functions provided by biological sensing devices are becoming more and more diverse, and the instance of transmitting medical information via a wireless/wired transmission way is becoming popular. Generally speaking, when a biological sensing device is being developed and designed, only specific biological sensing features are emphasized while other additional functions (e.g. a wireless/wired transmission function) are not the main consideration. Therefore, before a main printed circuit board on the biological sensing device leaves the factory, if modules or chips providing additional functions are not disposed on such printed circuit board, it is difficult for such biological sensing device to provide extra additional functions through mechanical fixes or modifications without changing the circuit architecture of the main printed circuit board of the biological sensing device after the printed circuit board and/or the biological sensing device leaves the factory.

### SUMMARY OF THE INVENTION

Accordingly, the present invention is directed to a multi-function biological sensing device which is able to provide extra additional functions without changing the circuit architecture of a main printed circuit board so as to improve the convenience in usage drastically.

According to an embodiment of the present invention, a multi-function biological sensing device is provided. The multi-function biological sensing device includes a multi-function card, a multi-function card slot, a first multi-function module, and a second multi-function module. The multi-function card includes a first pin set. The multi-function card slot is configured to receive the multi-function card and includes a second pin set. The multi-function module is disposed on the multi-function card and electrically connected to the first pin set. The second multi-function module is disposed on the multi-function card slot and electrically connected to the second pin set. Accordingly, the first multi-function module and the second multi-function module perform an electrical connection via the first pin set and the second pin set so as to provide at least one additional function.

According to an embodiment of the present invention, the multi-function biological sensing device further includes a control module. The control module is electrically connected to the second pin set and the second multi-function module and configured to determine if the first multi-function module and the second multi-function module perform the electrical connection via the first pin set and the second pin set. When the first multi-function module and the second multi-function module perform the electrical connection via the first pin set and the second pin set, the control module activates the at least one additional function provided by the first multi-function module via the second multi-function module.

According to an embodiment of the present invention, the control module includes a detecting circuit and a processor. The detecting circuit is electrically connected to the second pin set and configured to determine if the first multi-function module and the second multi-function module perform the electrical connection via the first pin set and the second pin set. The processor is electrically connected to the detecting circuit and the second multi-function module. When the first multi-function module and the second multi-function module perform the electrical connection via the first pin set and the second pin set, the detecting circuit transmits an enabling signal to the processor. Additionally, when the processor receives the enabling signal, the processor activates the at least one additional function provided by the first multi-function module via the second multi-function module.

According to an embodiment of the present invention, the at least one additional function includes a wireless transmission function.

According to an embodiment of the present invention, the first multi-function module includes an antenna and a wireless transmission chip. The wireless transmission chip is electrically connected to the antenna and configured to execute the wireless transmission function via a wireless communication protocol.

According to an embodiment of the present invention, the at least one additional function includes a wired transmission function.

According to an embodiment of the present invention, the first multi-function module includes a wired transmission interface configured to receive and transmit a wired transmission signal.

According to an embodiment of the present invention, the wired transmission interface includes a universal serial bus (USB) interface.

According to an embodiment of the present invention, the multi-function card is a password card configured to provide at least one set of password parameter and store the at least one set of password parameter in the multi-function biological sensing device.

According to an embodiment of the present invention, the multi-function card is a barcode scanning device configured to scan a barcode and store the scanned barcode in the multi-function biological sensing device.

According to an embodiment of the present invention, the second multi-function module is disposed corresponding to the first multi-function module.

To sum up, the first multi-function module and the second multi-function module are respectively disposed on the multi-function card and in the multi-function card slot of the multi-function biological sensing device in the present invention. When the first multi-function module and the second multi-function module perform the electrical connection via the first pin set and the second pin set, the first multi-function module and the second multi-function module may provide the at least one additional function.

Reference will now be made in detail to the present embodiments of the invention, examples of which are illustrated in the accompanying drawings. Wherever possible, the same reference numbers are used in the drawings and the description to refer to the same or like parts.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a partial cross-section diagram of a multi-function biological sensing device according to an embodiment of the present invention.

FIG. 2 is a schematic diagram of disposing pins according to an embodiment of the present invention.

FIG. 3 is a functional block diagram of a first multi-function module according to an embodiment of the present invention.

FIG. 4 is a functional block diagram of a wireless transmission chip according to an embodiment of the present invention.

FIG. 5 is a functional block diagram of the first multi-module according to another embodiment of the present invention.

FIG. 6 is a functional block diagram of a multi-function biological sensing device according to an embodiment of the present invention.

### DESCRIPTION OF THE EMBODIMENTS

In order not to restrict a biological sensing device (e.g. a blood glucose meter) to basic biological sensing functions (e.g. blood glucose measurement) but to provide one or a plurality of extra additional functions (e.g. a wireless/wired transmission function), a multi-function biological sensing device with multi-function modules corresponding to each other disposed in a multi-function card and a multi-function card slot respectively is provided in the embodiments of the present invention. When the multi-function card is inserted into the multi-function card slot, the multi-function modules corresponding to each other in the multi-function card and the multi-function card slot is able to be combined so as to provide at least one additional function (e.g. the wireless-wired transmission function). By that means, the flexibility and the variability in usage of the biological sensing device may be improved effectively in the present invention. Moreover, the present invention may also be combined with existing biological sensing devices/elements/components and provide high practicality.

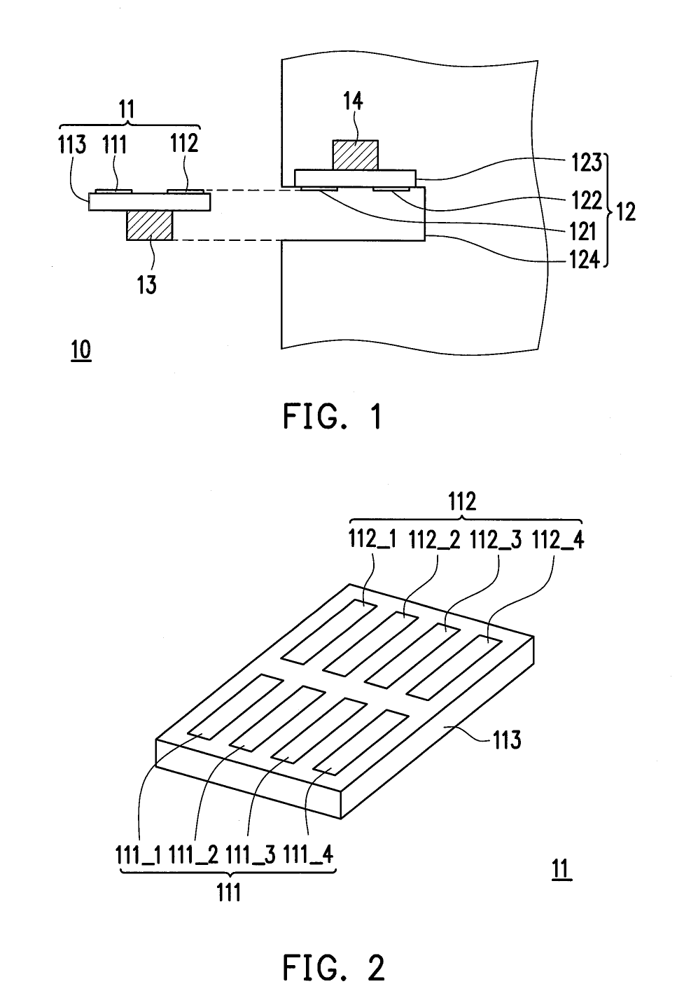

FIG. 1 is a partial cross-section diagram of a multi-function biological sensing device according to an embodiment of the present invention. Referring to FIG. 1, a multi-function biological sensing device 10 includes a multi-function card 11, a multi-function card slot 12, a first multi-function module 13, and a second multi-function module 14.

In the present embodiment, the multi-function card 11 includes pin sets 111 and 112 (or jointly referred to as a first pin set) and a printed circuit board 113. The pin sets 111 and 112 may be disposed on the printed circuit board 113 and realized by electrode sheets.

The multi-function card slot 12 is configured to receive the multi-function card 11 and includes pin sets 121 and 122 (or jointly referred to as a second pin set), a printed circuit board 123, and a card slot main body 124. Similarly, the pin sets 121 and 122 may be disposed on the printed circuit board 123 and realized by electrode sheets as well.

For example, FIG. 2 is a schematic diagram of disposing pins according to an embodiment of the present invention. Referring to FIG. 2, take the multi-function card 11 as an example. The pin set 111 on the multi-function card 11 may include pins 111_1-111_4, and the pin set 112 may include pins 112_1-112_4. Additionally, the way of disposing pins in the multi-function card slot (e.g. the pin sets 121 and 122) may be similar and corresponding to that of disposing the pin sets 111 and 112 and will not be repeated herein.

However, it should be understood that FIG. 2 is merely an embodiment of disposing the pins in the present invention. In practice, the number of the pins in the first pin sets and the second pin sets are not limited in the present invention. For example, in consideration of the cost and the practicality, the pin set 111 may be disposed on the multi-function card 11 in FIG. 2 alone without the pin set 112 being disposed in another embodiment of the present invention, wherein the number of the pins in the pin set 111 may be adjusted adaptively. Alternatively, in another embodiment, the disposition and the arrangement of each of the pin sets and/or pins may be adjusted according to the requirement of the practicality or the design.

Referring to FIG. 1 again, in the present embodiment, when the multi-function card 11 is inserted into the multi-function card slot 12, the first pin set (i.e. the pin sets 111 and 112) on the multi-function card 11 may be matched with and electrically connected to the second pin set (i.e. the pin sets 111 and 112) in the multi-function card slot 12. For example, the pin set 111 (including each of the pins therein) may contact with the pin set 121 (including each of the pins therein), and the pin set 112 (including each of the pins therein) may contact to the pin set 122 (including each of the pins therein). It is noted that each of the pins in the first pin set and the second pin set may include an individual function and feature, and the pins on each corresponding position on the first pin set and the second pin set include the function and feature corresponding to each other in the present embodiment.

The first multi-function module 13 is disposed on the multi-function card 11 and electrically connected to the first pin set (i.e. the pin sets 111 and 112). The first multi-function module 13 includes a necessary component providing one or a plurality of additional functions. The second multi-function module 14 is disposed within the multi-function card slot 12 and electrically connected to the second pin set (i.e. the pin sets 121 and 122). In the present embodiment, the second multi-function module 14 is disposed corresponding to the first multi-function module 13 and may be treated as a connector between the first multi-function module 13 and the multi-function biological sensing device 10. By that means, the first multi-function module 13 and the second multi-function module 14 may perform an electrical connection through the first pin set (i.e. the pin sets 111 and 112) and the second pin set (i.e. the pin sets 121 and 122) so as to provide the one or the plurality of additional functions to the multi-function biological sensing device 10.

Additionally, in an embodiment, when the additional function provided by the first multi-function module 13 is more than one, each of the pins electrically connected to each other on the first pin set (i.e. the pin sets 111 and 112) and the second pin set (i.e. the pin sets 121 and 122) may transmit data or signals corresponding to the additional function individually. In other words, the additional function, the transmitted signal, and the operating method corresponding to each of the pins in the embodiments of the present invention may be set based on the requirement of the practicality or the design and are not limited in the present invention.

Take FIG. 2 as an example. Assume that the additional function corresponding to the pin 111_1 is the wireless transmission function. When the multi-function card is inserted into the multi-function card slot 12, the pin 111_1 may transmit wireless data to be transmitted or to be received via the pin electrically connected to the pin 111_1 on the pin set 121. However, in an embodiment, it is possible that the plurality of pins correspond to the same additional function, and the present invention is not limited thereby.

In the present embodiment, the aforementioned additional functions may be, for example, the wireless transmission function and/or the wired transmission function provided by the first multi-function module 13. By that means, the biological sensing device 10 may use the wireless transmission function and/or the wired transmission function provided by the first multi-function module 13 via the second multi-function module 14 to perform communication with other electronic devices with wireless/wired transmission function such as a computer, a smart phone, or a tablet computer.

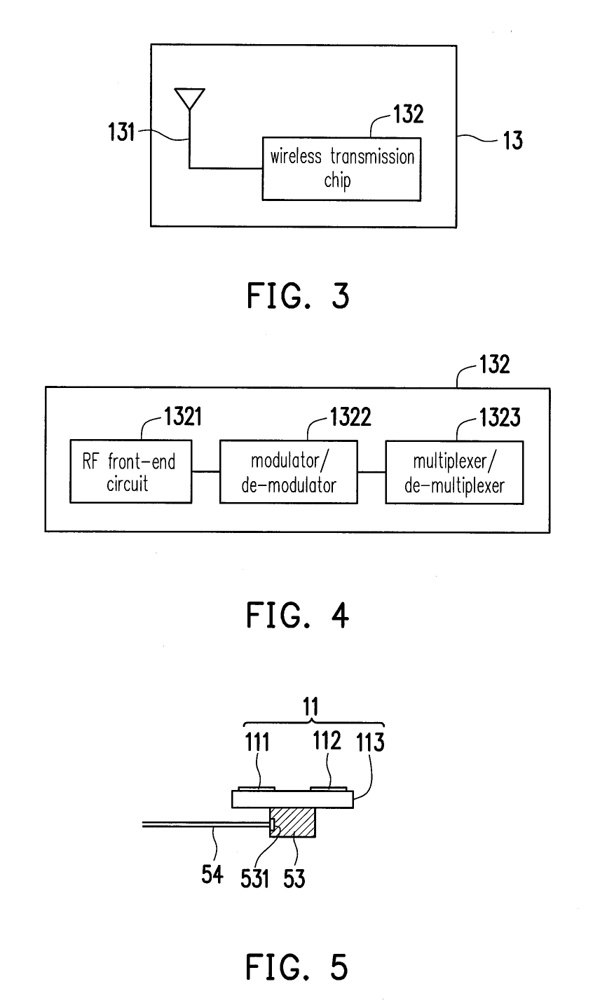

For example, FIG. 3 is a functional block diagram of a first multi-function module according to an embodiment of the present invention. Referring to FIG. 3, take the first multi-function module 13 as an example. The first multi-function module 13 may include an antenna 131 and a wireless transmission chip 132. The antenna 131 is configured to receive or transmit wireless signals. The wireless transmission chip 132 is electrically connected to the antenna 131 and configured to perform the wireless transmission function via a wireless communication protocol.

For example, FIG. 4 is a functional block diagram of a wireless transmission chip according to an embodiment of the present invention. Referring to FIG. 4, take the wireless transmission chip 132 as an example. The wireless transmission chip 132 may include a radio frequency (RF) front-end circuit 1321, a modulator/de-modulator 1322, and a multiplexer/de-multiplexer 1323.

For example, when the antenna 131 receives a wireless signal transmitted from a remote wireless communication device, the RF front-end circuit 1321 may convert the wireless signal to a medium frequency (MF) signal or an in-phase/quadrature signal. Then, the modulator/de-modulator 1322 may de-modulate the MF signal or the in-phase/quadrature signal to a transport stream. Then, the multiplexer/de-multiplexer 1323 may perform de-multiplexing on the transport stream so as to recognize effective packets from the transport stream and transmit the effective packets to a processor of the biological sensing device 10 via the second multi-function module 14.

Additionally, when the biological sensing device 10 wishes to transmit data (e.g. according to an operation of a user) to the remote wireless communication device, the processor of the biological sensing device 10 may first transmit the data to be transmitted to the wireless transmission chip 132 via the second multi-function module 14. Then, the wireless transmission chip 132 may encapsulate the data to be transmitted into packets, and the multiplexer/de-multiplexer 1323 performs multiplexing on the packets so as to generate a transport stream. Afterwards, the modulator/de-modulator 1322 may modulate the transport stream to a MF signal or an in-phase/quadrature signal. Lastly, the RF front-end circuit 1321 may transmit the MF signal or the in-phase/equdrature signal to the remote wireless communication device via the antenna 131.

Additionally, the wireless communication protocol may be the 3^{rd} Generation (3G) of mobile telecommunications technology, the Bluetooth, the General Packet Radio Service (GPRS), the High Speed Downlink Packet Access (HSDPA), the High Speed Uplink Packet Access (HSUPA), the Wireless-Fidelity (Wi-Fi), the Wireless Wide Area Network (WWAN), the Zigbee, and the 3^{rd} Generation Partnership Project Long Term Evolution (3GPP LTE), and yet the present invention is not limited thereto. Additionally, the aforementioned realization of the first multi-function module 13 is merely an exemplary embodiment, and the present invention is not limited thereby.

For example, FIG. 5 is a functional block diagram of the first multi-module according to another embodiment of the present invention. Referring to FIG. 5, take the first multi-function module 53 as an example. The multi-function module 53 may include a wireless transmission interface 531, and it may receive a wired signal from a transmission line 54 via the wired transmission interface 53 or transmit a wired signal to the transmission line 54 so as to perform the wired transmission function. For example, in the present embodiment, the wired transmission interface 531 is, for example, a universal serial bus (USB) interface, a phone jack, or a RS232 interface. Additionally, the USB interface may be, for example, a standard USB interface, a mini USB interface, or a micro USB interface. The pins 111 and 112 on the multi-function card 11 and the printed circuit board 113 are described in detail in FIG. 1 and will not be repeated herein.

Alternatively, the first multi-function module 13 and the second multi-function module 14 may respectively provide a portion of components required for an additional function so as to provide such additional function by combining the first multi-function module 13 and the second multi-function module 14. For example, in an embodiment, the first multi-function module 13 includes the antenna 131, and the second multi-function module 14 includes the wireless transmission chip 132. After the first multi-function module 13 and the second multi-function module 14 are electrically connected via the first pin set and the second pin set, the first multi-function module 13 and the second multi-function module 14 may jointly provide the wireless transmission function.

It is noted that a biological sensing device (e.g. the multi-function biological sensing device 10) with the wireless/wired transmission function activated may be treated as a gateway. For example, when a biological sensing device without wireless/wired transmission function is connected to the multi-function biological sensing device 10 with the wireless transmission function activated via a connecting line, the biological sensing device without wireless/wired transmission function may perform the wireless/wired transmission function or the other additional functions provided by the multi-function biological sensing device 10 via the multi-function biological sensing device 10. By that means, the additional functions provided by multi-function biological device 10 in the present invention may be effectively shared to more biological sensing devices or related electronic devices without each of the additional functions (e.g. the wireless/wired transmission function).

Additionally, the first multi-function module and the second multi-function module provided in the present invention may be combined with an existing product. For example, in an embodiment, the multi-function card 11 is, for example, a password card, which is configured to provide at least a set of password parameter to the multi-function biological sensing device 10. For example, the password parameter may be a correcting parameter corresponding to a sample strip and stored in a memory of the multi-function card 11.

In an embodiment, assume that the multi-function card 11 is a password card, and the multi-function card slot 12 is a password card slot. When the user inserts the multi-function card (i.e. the password card) into the multi-function card slot 12 (i.e. the password card slot), the first multi-function module 13 on the multi-function card 11 may transmit and store the aforementioned password parameter in the multi-function biological sensing device 10 via the second multi-function module 14 on the multi-function card slot 12. Then, when the user inserts a sample strip with a biological characteristic (e.g. saliva, blood, or urine) into a sample slot (unillustrated) so as to execute a biological sensing function (e.g. a blood glucose measurement, an uric acid measurement, a cholesterol measurement, or a pH value measurement), the multi-function biological sensing device 10 may obtain the corresponding password parameter from the stored password parameter according to the lot number, the serial number or other related information of the sample strip so as to perform testing and correcting on the sample strip.

Alternatively, in an embodiment, when the multi-function biological sensing device 10 requires to perform testing and correcting on the sample strip, the multi-function biological sensing device 10 may read the required password parameter from the memory of the multi-function card 11 via the first multi-function module 13 and the second multi-function module 14 in real time. Then, the multi-function biological sensing device 10 may perform testing and correcting on the sample strip according to the obtained password parameter.

However, the present invention is not limited to the aforementioned realization. In another embodiment, the multi-function card 11 may also be a barcode reader configured to scan and read barcodes. In other words, in the present embodiment, the additional functions provided by the first multi-function module 13 on the multi-function card 11 may include a barcode scanning and/or transmitting function. Afterwards, the barcode scanning device (i.e. the multi-function card 11) may transmit the scanned barcode to the multi-function card slot 12 via the pin corresponding to the barcode transmitting function and then to the processor of the multi-function biological sensing device 10 via the second multi-function module 14 so that the processor of the multi-function biological sensing device 10 may store the scanned barcode so as to perform followed-up applications.

In other words, a plurality of additional functions are able to be integrated into the first multi-function module, and the data and the signal of the additional function corresponding to each of the pins may be transmitted via different pins simultaneously in the present invention. Additionally, the biological sensing device in the present invention may provide a more variety of additional functions by inserting different types of multi-function cards as well as changing multi-function cards and the second multi-function module in the multi-function card slot simultaneously.

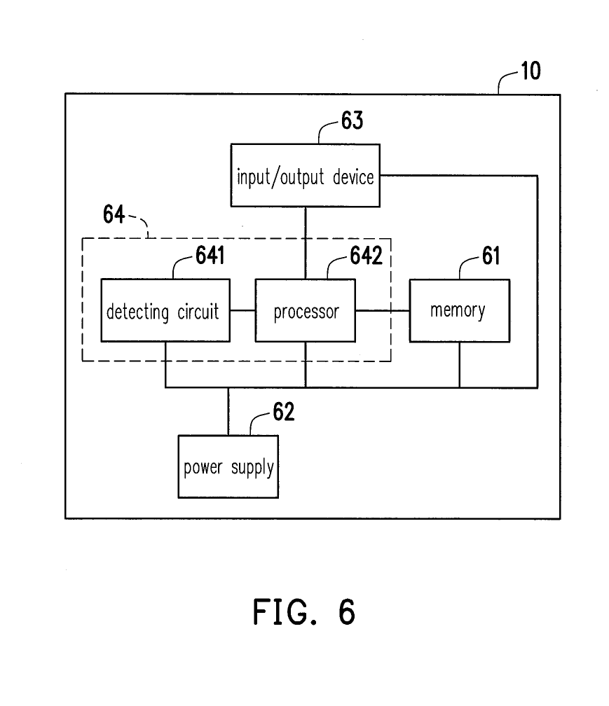

In another aspect, FIG. 6 is a functional block diagram of a multi-function biological sensing device according to an embodiment of the present invention. Referring to both FIG. 1 and FIG. 6, the multi-function biological sensing device 10 includes a memory 61, a power supply 62, an input/output device 63, and a control module 64 in the present embodiment.

The memory 61 is configured to store data. In the present embodiment, the memory 61 is, for example, each non-volatile memory or the combination thereof such as a read-only memory (ROM) and/or a flash memory.

The power supply 62 is configured to provide the power for the multi-function biological sensing device 10 to operate. The power supply 62 may be, for example, a battery. The input/output device 63 may be a signal input/output device such as a touch screen, a button, a mouse, a earphone, a microphone, or a speaker.

The control module 64 is electrically connected to the second pin set (i.e. the pin sets 121 and 122) and the second multi-function module 14 and configured to determine if the first multi-function module 13 and the second multi-function module 14 performs the electrical connection via the first pin set (i.e. the pin sets 111 and 112) and the second pin set (i.e. the pin sets 121 and 122). When the first multi-function module 13 and the second multi-function module 14 perform the electrical connection via the first pin set (i.e. the pin sets 111 and 112) and the second pin set (i.e. the pin sets 121 and 122), it represents that the multi-function card 11 is inserted in the multi-function card slot 12. Meanwhile, the control module 64 may activate or enable one or a plurality of additional functions provided by the first multi-function module 13 via the second multi-function module 14.

In the present embodiment, the control module 64 may also include a detecting circuit 641 and a processor 642. The detecting circuit 641 is electrically connected to the second pin set (i.e. the pin sets 121 and 122) and configured to determine if the first multi-function module 13 and the second multi-function module 14 performs the electrical connection via the first pin set (i.e. the pin sets 111 and 112) and the second pin set (i.e. the pin sets 121 and 122). For example, in the present embodiment, the detecting circuit 641 may determine if the first multi-function module 13 and the second multi-function module 14 performs the electrical connection via the first pin set (i.e. the pin sets 111 and 112) and the second pin set (i.e. the pin sets 121 and 122) according to an on/off status of the one or the plurality of pins on the second pin set (i.e. the pin sets 121 and 122). Then, when the detecting circuit 641 determines that the first multi-function module 13 and the second multi-function module 14 are electrically connected via the first pin set (i.e. the pin sets 111 and 112) and the second pin set (i.e. the pin sets 121 and 122), the detecting circuit 641 may transmit an enabling signal to the processor 642.

The processor 642 is electrically connected to the detecting circuit 641 and the second multi-function module 14. In the present embodiment, the processor 642 may be a micro-processor or a central processing unit (CPU), and the present invention is not limited thereto.

When the processor 642 receives the enabling signal from the detecting circuit 641, the processor 642 may activate or enable the one or the plurality of additional functions provided by the first multi-function module 13 via the second multi-function module 14 and process the data related to the additional functions (e.g. packet data to be received or transmitted or barcode data). For example, in an embodiment, when the processor 642 receives the enabling signal from the detecting circuit 641, the processor 642 may activate or enable the wired/wireless transmission function provided by the first multi-function module 13 via the second multi-function module 14 so as to receive or transmit data via the wired/wireless transmission function.

To sum up, the present invention provides one or a plurality of additional function so as to effectively improve the limitation in usage of conventional biological sensing devices (e.g. data transmission) by disposing additional multi-function modules on a multi-function card and a multi-function card slot. In terms of the functions (e.g. a wireless/wired transmission function) excluding from a main circuit board upon manufacture, it is noted that the circuit structure of the main circuit board is not require to be adjusted, and only the multi-function modules may be disposed in a form of mechanical components on the multi-function card (e.g. a password card) and the multi-function card slot (e.g. a password card slot) in the present invention. Therefore, the practicality and the convenience in usage are drastically improved.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A multi-function biological sensing device comprising:
a multi-function card, comprising a first pin set;
a multi-function card slot, configured to receive the multi-function card and including a second pin set;
a first multi-function module, disposed on the multi-function card and electrically connected to the first pin set; and
a second multi-function module, disposed on the multi-function card slot and electrically connected to the second pin set,
wherein the first multi-function module and the second multi-function module perform an electrical connection via the first pin set and the second pin set so as to provide at least one additional function.

2. The multi-function biological sensing device of claim 1 further comprising:
a control module, electrically connected to the second pin set and the second multi-function module, and configured to determine if the first multi-function module and the second multi-function module perform the electrical connection via the first pin set and the second pin set,
wherein when the first multi-function module and the second multi-function module perform the electrical connection via the first pin set and the second pin set, the control module activates the at least one additional function provided by the first multi-function module via the second multi-function module.

3. The multi-function biological sensing device of claim 2, wherein the control module comprises:
a detecting circuit, electrically connected to the second pin set, and configured to determine if the first multi-function module and the second multi-function module perform the electrical connection via the first pin set and the second pin set; and
a processor, electrically connected to the detecting circuit and the second multi-function module,
wherein when the first multi-function module and the second multi-function module perform the electrical connection via the first pin set and the second pin set, the detecting circuit transmits an enabling signal to the processor,
wherein when the processor receives the enabling signal, the processor activates the at least one additional function provided by the first multi-function module via the second multi-function module.

4. The multi-function biological sensing device of claim 1, wherein the at least one additional function comprises a wireless transmission function.

5. The multi-function biological sensing device of claim 4, wherein the first multi-function module comprises:
an antenna; and
a wireless transmission chip, electrically connected to the antenna, and configured to perform the wireless transmission function via a wireless communication protocol.

6. The multi-function biological sensing device of claim 1, wherein the at least one additional function comprises a wired transmission function.

7. The multi-function biological sensing device of claim 6, wherein the first multi-function module comprises a wired transmission interface, configured to receive and transmit a wired transmission signal.

8. The multi-function biological sensing device of claim 7, wherein the wired transmission interface comprises a universal serial bus (USB) interface.

9. The multi-function biological sensing device of claim 1, wherein the multi-function card is a password card, configured to provide at least one set of password parameter and store the at least one set of password parameter in the multi-function biological sensing device.

10. The multi-function biological sensing device of claim 1, wherein the multi-function card is a barcode scanning device, configured to scan a barcode and store the scanned barcode in the multi-function biological sensing device.

11. The multi-function biological sensing device of claim 1, wherein the second multi-function module is disposed corresponding to the first multi-function module.
